(19) United States
(12) Patent Application Publication
(10) Pub. No.: US 2015/0208914 A1
(43) Pub. Date: Jul. 30, 2015

(54) APPARATUS AND METHOD FOR DETERMINING VISUAL ACUITY OF A SUBJECT

(71) Applicant: UNIVERSITY OF DURHAM, Durham, Durham (GB)

(72) Inventors: Gordon Derek Love, Richmond (GB); Martin Scott Banks, Berkeley, CA (US); Simon Berry, Durham (GB)

(21) Appl. No.: 14/423,044

(22) PCT Filed: Aug. 21, 2013
(86) PCT No.: PCT/GB2013/052205

§ 371 Date: Feb. 20, 2015

(30) Foreign Application Priority Data
Aug. 24, 2012 (GB) 1215117.1

(51) Int. Cl. A61B 3/036; A61B 3/00; A61B 3/028; A61B 3/032

(52) U.S. Cl. CPC A61B 3/036; A61B 3/032; A61B 3/0025; A61B 3/0285

(57) ABSTRACT

An apparatus (2) for providing a focussed visual state of a subject is disclosed. The apparatus comprises a fast focus switchable lens (6) for alternately focusing an image at (i) a first position at a first distance behind a second position and at (ii) a third position at a second distance in front of the second position, wherein the first distance is substantially equal to the second distance. A refraction correction lens system (12) adjusts the second position.

The present invention relates to an apparatus and method for providing a focussed visual state of a subject. The invention relates particularly, but not exclusively, to an apparatus and method for determining necessary visual correction for a patient.

Refractive errors in the eye are extremely common and from the age of about 45 almost everyone requires some type of visual aid. As part of a standard eye test the patient is required to read letters on a Snellen chart, and different lenses (known as trial lenses to persons skilled in the art) are used to assess the optimum correction.

Studies have investigated the blur discrimination threshold of the human eye, with results ranging from 0.02 D up to 1.75 D (dioptres). It has been determined that from a magnitude of 0.87 D, the smallest noticeable change in blur was substantially the same, up until the point where an image could no longer be recognised. Research on edge detection and recognition provided information to the effect that a change in blur is easier to determine for out of focus images.

Known methods and apparatus for determining the necessary optical correction for a patient involve focussing an image on a retina of a patient. Such methods and apparatus suffer from the drawback that the accuracy with which the focussed condition of the image can be determined is limited.

Preferred embodiments of the present invention seek to overcome the above disadvantage of the prior art.

According to an aspect of the present invention, there is provided an apparatus for providing a focussed visual state of a subject, the apparatus comprising:

focusing means for alternately focusing at least one image at (i) a respective first position at a respective first distance behind a respective second position and at (ii) a respective third position at a respective second distance in front of said respective second position, wherein said first distance is substantially equal to said second distance; and

first adjustment means for adjusting said respective second position.

The present invention is based on the finding that the human eye is more sensitive to the detection of flickering images than to the blurring of a static image. By providing focusing means for alternately focusing an image at a first position at a first distance behind a second position and at a third position at a second distance in front of the second position, wherein the first distance is substantially equal to the second distance, and

first adjustment means for adjusting the second position, this provides the advantage that a focussed condition of an image, and therefore any necessary refractive correction for a patient, can be more accurately determined by determining the second position for which cessation of flicker between the alternating images occurs. This also provides the advantage that a “subjective refractive” method of determining focus, in which the position of optimum focus is determined by the patient, is provided, which will generally be more familiar to patients than alternative “objective refractive” methods.

The focusing means may comprise a focusing portion adapted to alternately focus at least one said image at the respective first and third positions, and selection means for selecting focusing between said respective first and third positions.

The focusing portion may comprise a birefringent lens.

The selection means may include at least one liquid crystal device for adjusting the plane of polarisation of incident light.

By providing at least one liquid crystal device for adjusting the plane of polarisation of incident light, this provides the advantage of enabling rapid switching between focal states.

The first adjustment means may be adapted to be activated by a subject.

The apparatus may further comprise output means for determining a distance of said respective second position from a retina of the subject.

The apparatus may further comprise second adjustment means for locating said respective second position at a retina of the subject.

The apparatus may further comprise magnification adjustment means for adjusting the magnification of an image focussed at said respective first position compared with an image focussed at said respective third position.

This provides the advantage of enabling errors of magnification between the images to be corrected, to ensure that flicker minimisation correctly corresponds to the second position being located at a retina of the subject.

The magnification adjustment means may be adapted to focus a plane of said focussing means onto a pupil of an eye of a subject.

The focusing means may be adapted to alternately focus at least three different images at respective first and third positions.

This provides the advantage of enabling a degree of astigmatism to be determined.

The apparatus may further comprise processing means for determining a degree of astigmatism from said respective second positions.

According to another aspect of the present invention, there is provided a computer program product executable on a computer for determining a focussed visual state of a subject, the computer program product comprising;

first computer code executable for controlling focusing means for alternately focusing at least one image at (i) a respective first position at a respective first distance behind a respective second position and at (ii) a respective third position at a respective second distance in front of said respective second position, wherein said first distance is substantially equal to said second distance; and

second computer code executable for controlling first adjustment means for adjusting said respective second position.

The computer program product may further comprise third computer code executable for determining distance of said respective second position from a retina of a subject.

The computer program product may further comprise fourth computer code executable to control second adjustment means for locating said respective second position at a retina of the subject.

The first computer code may be executable for controlling focusing means to alternately focus at least three different images at respective first and third positions.

The computer program product may further comprise fifth computer code executable to determine a degree of astigmatism from said respective second positions.

According to a further aspect of the present invention, there is provided a method for providing a focussed visual state of a subject, the method comprising alternately focusing at least one image at (i) a respective first position at a respective first distance behind a respective second position and at (ii) a respective third position at a respective second distance in front of said respective second position, wherein said first distance is substantially equal to said second distance; and

adjusting said respective second position.

The method may further comprise determining a distance of said respective second position from a retina of the subject.

The method may further comprise locating said respective second position at a retina of the subject.

The method may further comprise adjusting the magnification of an image focussed at said respective first position compared with an image focussed at said respective third position.

The magnification adjustment may comprise focussing a plane of said focussing means onto a pupil of an eye of a subject.

The method may further comprise alternately focussing at least three different images at respective first and third positions.

The method may further comprise determining a degree of astigmatism from said respective second positions.

A preferred embodiment of the invention will now be described, by way of example only and not in any limitative sense, with reference to the accompanying drawings, in which:

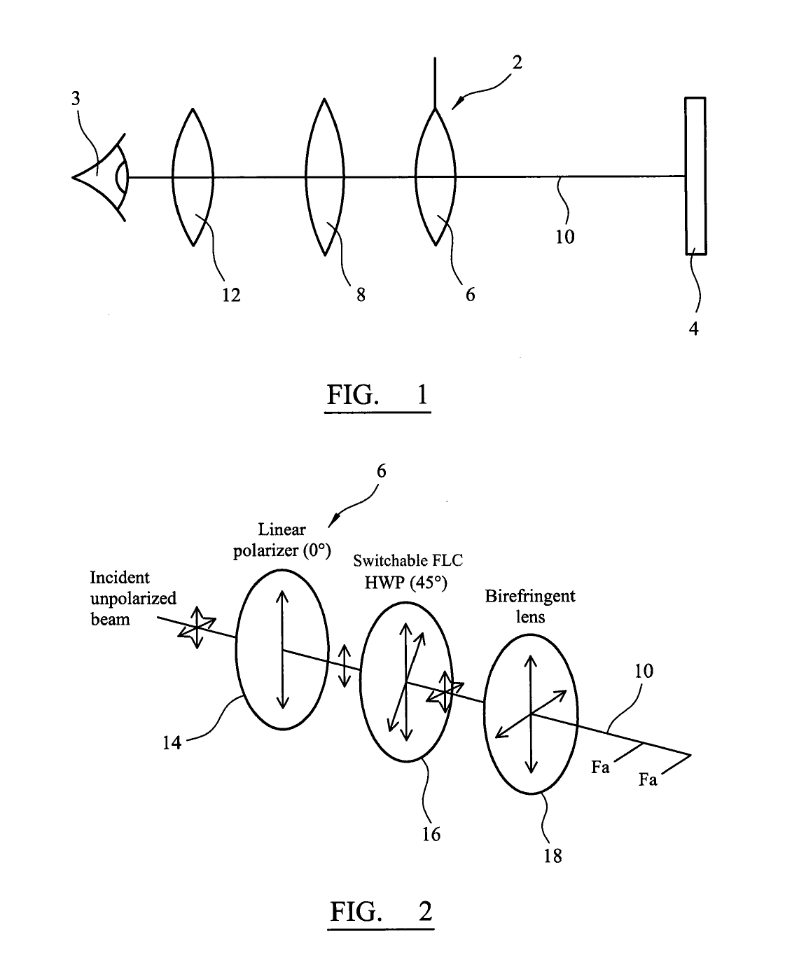

FIG. 1 is a schematic view of an apparatus embodying the present invention for determining visual correction necessary for a patient;

FIG. 2 is a schematic view of a focus switchable lens of the apparatus of FIG. 1;

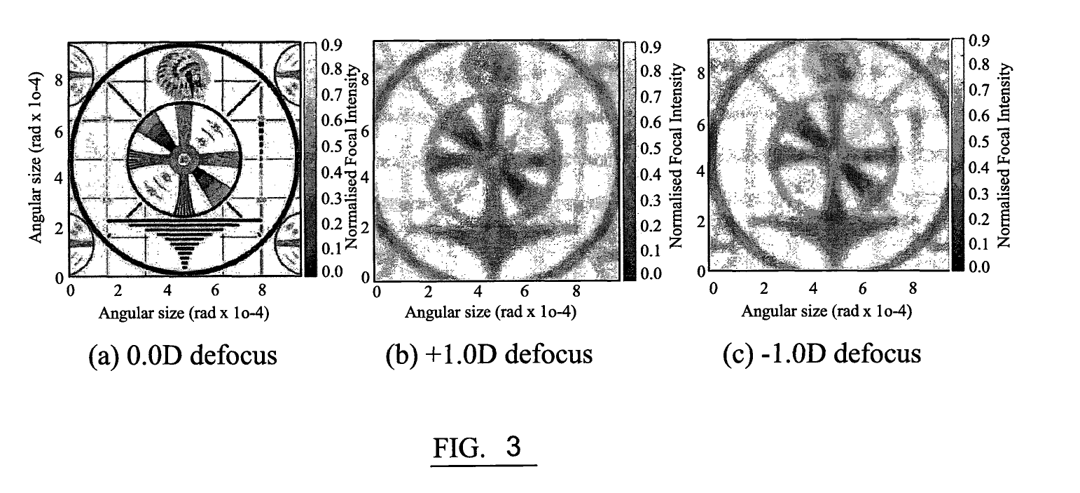

FIG. 3 is an illustration of simulated images with equal but opposite defocus errors;

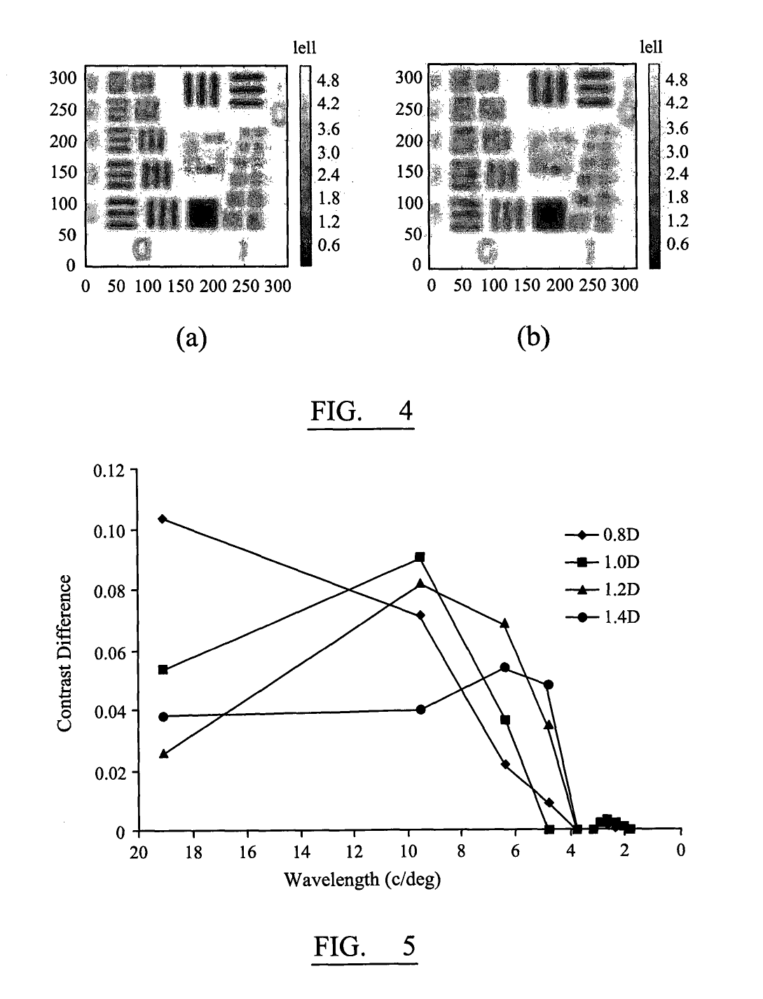

FIG. 4 shows two images of a defocused test pattern; and

FIG. 5 illustrates the effect of spatial frequency and base line defocus on image contrast difference.

Referring to FIG. 1, an apparatus 2 for determining visual correction necessary for a patient (of which only an eye 3 is shown in FIG. 1) comprises an illuminated display in the form of a Snellen chart 4, focussing means in the form of a fast focus switchable lens 6 (which will be described in greater detail with reference to FIG. 2), and a reimaging lens system 8 to refocus the plane of the switchable lens 6 onto the exit pupil of the eye 3 to ensure no magnification is seen when the switchable lens 6 is activated. An image at the display 4 is shown such that the eye 3 of a patient views the display 4 along optical axis 10 of the apparatus 2, and the focus of a refraction correcting lens system 12 (which may be just different pairs of spectacles, or a phoropter, or a variable lens) is adjusted in a manner described in greater detail below.

Referring to FIG. 2, the focus switchable lens 6 comprises a linear polariser 14 for generating plane polarised light, a ferroelectric liquid crystal device 16 which switches the state of polarisation between two polarisation states at right angles to each other and a birefringent lens 18 having different focal lengths for the two different polarisation states. Accordingly, the focus switchable lens 6 can be switched between a first state of focal length Fₑ and a second state of focal length Fₒ.

Referring again to FIG. 1, the reimaging lens system 8 is arranged so that, optically, the plane of the switchable lens 6 is in the same plane as the lens of the eye 3 since otherwise a switch in focal length will cause a change in magnification of the image on the display 4. The reimaging lens system 8 may also contain a lens to remove some of the optical power of the switchable lens 6 if there is a fixed offset which is too large, as will be appreciated by persons skilled in the art.

In order to explain the principle of operation of the present invention, the focus switchable lens 6 firstly switches at a frequency of the typically about 10 Hz between a positive and negative focus error, for example +1 D and −1 D. If the refraction correcting lens system 12 is perfectly adjusted so that the patient has optimum correction, and the patient has no higher order aberrations, then the focus switchable lens 6 will focus the image between states having a refractive error of +1 D and −1 D. If the focus switchable lens 6 is arranged to be in the conjugate focal plane to the pupil of the patient's eye 3, then the two resulting images have the same magnification and therefore appear the same. If, however, the lens system 12 is adjusted by a small amount, say +0.1 D, then the total refractive error will be switched rapidly between +1.1 D and −0.9 D, and the patient will see a rapidly flickering image. It has been discovered that the patient will see this flickering much more clearly than a simple change in focus from 0.0 D to 0.1 D produced by manually tuning the system, thereby enabling more accurate determination of the correct focal point of a spectacle system.

Referring to FIG. 3, simulated images are shown which illustrate that two images with equal but opposite defocus errors are identical. Accordingly, the focus switchable lens 6 is used to switch between equal and opposite amounts of defocus by placing an object between the two points of focus. The position of the object is dependent on the refraction of the patient looking through the focus switchable lens 6.

Blur discrimination was tested using a series of simulated videos consisting of two images shown alternately at a set frequency, where each image contained a different magnitude of defocus. Defocus is defined by a standard Zernike polynomial term Z₂⁰(ρ,θ)=2ρ²−1, in polar coordinates, where ρ is the radial coordinate and θ the angular coordinate. This was used to describe a pupil function P(ρ)=exp[−i2πAZ₂⁰], where A denotes the magnitude of the defocus in waves. This was Fourier transformed and squared to give a point spread function (PSF) which was then convolved with an object to determine the image. The pixel scale of the PSF governs the angular size of the final image and is defined as

$$ν = \frac{λ}{α W} ,$$

where λ is the wavelength, a the over sampling scale and W the eye's pupil size. An image of size m×m pixels will therefore have an angular size of my and the viewing distance for the images created is therefore s/v where s is the size of the image.

A series of videos was then created to test the effect of spatial frequency, temporal frequency and baseline defocus on blur discriminability. The individual images used to create the video are shown in FIG. 4.

In order to demonstrate flicker experimentally, a camera was used in conjunction with an optical system containing a focus switchable lens as in FIG. 1. The birefringent lens is placed directly in front of a camera lens to prevent any change in image magnification caused by the focus switchable lens. A zoom lens system is used so that the point of equi-blur can be determined by the patient using an actuator to adjust the zoom lens system. This arrangement was used to test the precision with which a patient can find the point of equi-blur compared with the accuracy of finding perfect focus without flicker. The patient was then required to utilise the actuator, while viewing the camera video feed, to readjust the zoom lens system back to the point of equal blur. This process was repeated several times, and the same procedure was carried out with flicker turned off.

| | Standard Deviation of Lens Position (mm) | | | |
|---|---|---|---|---|
| Grid Square | No Flicker | | Flicker | |
| Size (pixels) | Subject 1 | Subject 2 | Subject 1 | Subject 2 |
| 16 | 0.34 ± 0.10 | 0.39 ± 0.12 | 0.24 ± 0.08 | 0.23 ± 0.07 |
| 40 | 0.50 ± 0.15 | 0.55 ± 0.17 | 0.56 ± 0.18 | 0.40 ± 0.13 |

The standard deviation values shown in table 1 indicate the precision with which subjects were able to position the zoom lens system. The table indicates that the use of flicker improves defocus discrimination in 3 out of the 4 situations.

The above method is based on the assumption that the only error in the patient's eye is defocus (known as sphere in ophthalmology). However, many people also suffer from astigmatism, as a result of which the image will still flicker when in the optimum focus position using the above method. Some patients may also have higher order aberrations which, even though these are generally not measured, will also affect the result.

In an alternative embodiment, astigmatism can also be measured. Astigmatism generally means that the eye has two different focal lengths for two different orientations of structure in the image. For example, horizontal lines may be focused at one distance, whereas vertical lines may be focused at a different distance. As a result, an ophthalmic test will need to measure three items of data, i.e. the dioptric defocus value (called sphere) the dioptric astigmatic value, and the angle or orientation of the astigmatism.

In the alternative embodiment, the apparatus described above is used to carry out measurements on three separate patterns, i.e. bars orientated at 0°, 45° and 90° (or three other non-degenerate values). Using the above method, three different defocus values are obtained, and these three different values of defocus can then be used to calculate the required values of sphere, astigmatism and angle of astigmatism.

By way of further explanation of the above method, any wavefront aberration can be decomposed into a series of Zernike aberrations. In the present case, only defocus and astigmatism are of interest (which consists of two differently oriented Zernike terms). The wavefront aberration of the eye, W is therefore given by

W(r,θ)=z₃(2r²−1)+z₄r² cos 2θ+z₅r² sin 2θ

where r and θ are the polar coordinates, and z₃, z₄ and z₅ are the Zernike coefficients to be measured for defocus, and the two astigmatism terms. On viewing vertical bars, the z₅ term is not relevant because as switching between two different focal positions occurs, this mode will not change the resultant images. Similarly, when viewing bars oriented at 45°, the z₄ term is not important. Three measurements of defocus are then carried out, which are denoted as H (for horizontal bars), V (for vertical bars), and F (for bars at 45°). Omitting constants of proportionality and calibration, it can then be seen that,

H=z₃+z₄,

V=Z₃−Z₄,

F=z₃+z₅.

These three equations can then be solved to give the Zernike coefficients, z₃, z₄ and z₅, as will be familiar to persons skilled in the art. It will also be appreciated by persons skilled in the art that bars oriented at angles other than 0°, 45° and 90° can be used in order to solve the simultaneous equations set out above.

It will be appreciated by persons skilled in the art that the above embodiment has been described by way of example only and not in any limitative sense, and that various alterations and modifications are possible without departure from the scope of the invention as defined by the appended claims.

1. An apparatus for providing a focussed visual state of a subject, the apparatus comprising:
at least one focusing device for alternately focusing at least one image at (i) a respective first position at a respective first distance behind a respective second position and at (ii) a respective third position at a respective second distance in front of said respective second position, wherein said first distance is substantially equal to said second distance; and
at least one first adjustment device for adjusting said respective second position.

2. An apparatus according to claim 1, wherein said at least one focusing device comprises a focusing portion adapted to alternately focus at least one said image at the respective first and third positions, and at least one selection device for selecting focusing between said respective first and third positions.

3. An apparatus according to claim 2, wherein said focusing portion comprises a birefringent lens.

4. An apparatus according to claim 2, wherein at least one said selection device includes at least one liquid crystal device for adjusting the plane of polarisation of incident light.

5. An apparatus according to claim 1, wherein at least one said first adjustment device is adapted to be activated by a subject.

6. An apparatus according to claim 1, further comprising at least one output device for determining a distance of said respective second position from a retina of the subject.

7. An apparatus according to claim 1, further comprising at least one second adjustment device for locating said respective second position at a retina of the subject.

8. An apparatus according to claim 1, further comprising at least one magnification adjustment device for adjusting the magnification of an image focussed at said respective first position compared with an image focussed at said respective third position.

9. An apparatus according to claim 8, wherein at least one said magnification adjustment device is adapted to focus a plane of at least one said focussing device onto a pupil of an eye of a subject.

10. An apparatus according to claim 1, wherein at least one said focusing device is adapted to alternately focus at least three different images at respective first and third positions.

11. An apparatus according to claim 10, further comprising at least one processing device for determining a degree of astigmatism from said respective second positions.

12. A computer program product executable on a computer for determining a focussed visual state of a subject, the computer program product comprising;
first computer code executable for controlling at least one focusing device for alternately focusing at least one image at (i) a respective first position at a respective first distance behind a respective second position and at (ii) a respective third position at a respective second distance in front of said respective second position, wherein said first distance is substantially equal to said second distance; and
second computer code executable for controlling at least one first adjustment device for adjusting said respective second position.

13. A computer program product according to claim 12, further comprising third computer code executable for determining distance of at least one said respective second position from a retina of a subject.

14. A computer program product according to claim 12, further comprising fourth computer code executable to control at least one second adjustment device for locating said respective second position at a retina of the subject.

15. A computer program product according to claim 12, wherein said first computer code is executable for controlling at least one focusing device to alternately focus at least three different images at respective first and third positions.

16. A computer program product according to claim 15, further comprising fifth computer code executable to determine a degree of astigmatism from said respective second positions.

17. A method for providing a focussed visual state of a subject, the method comprising alternately focusing at least one image at (i) a respective first position at a respective first distance behind a respective second position and at (ii) a respective third position at a respective second distance in front of said respective second position, wherein said first distance is substantially equal to said second distance; and
adjusting said respective second position.

18. A method according to claim 17, further comprising determining a distance of said respective second position from a retina of the subject.

19. A method according to claim 17, further comprising locating said respective second position at a retina of the subject.

20. A method according to claim 17, further comprising adjusting the magnification of an image focussed at said respective first position compared with an image focussed at said respective third position.

21. A method according to claim 20, wherein said magnification adjustment comprises focusing a plane of at least one said focussing device onto a pupil of an eye of a subject.

22. A method according to claim 17, further comprising alternately focussing at least three different images at respective first and third positions.

23. A method according to claim 22, further comprising determining a degree of astigmatism from said respective second positions.
